(19) United States
(12) Patent Application Publication
(10) Pub. No.: US 2014/0371704 A1
(43) Pub. Date: Dec. 18, 2014

(54) PANTS-TYPE DIAPER AND CORRESPONDING MANUFACTURING PROCESS

(71) Applicant: Fameccanica.Data S.p.A., Sambuceto di San Giovanni Teatino (IT)

(72) Inventors: Gabriele SABLONE, Montesilvano (Pescara) (IT); Giacomo DE LAURETIS, Martinsicuro (Teramo) (IT)

(21) Appl. No.: 14/475,086

(22) Filed: Sep. 2, 2014

Related U.S. Application Data
(63) Continuation of application No. 12/914,422, filed on Oct. 28, 2010.
(60) Provisional application No. 61/256,163, filed on Oct. 29, 2009.

(51) Int. Cl. A61F 13/56; A61F 13/49; A61F 13/62

(52) U.S. Cl. CPC A61F 13/5655; A61F 13/622; A61F 13/4902; A61F 2013/49028; A61F 2013/49044; A61F 2013/49049; A61F 2013/49088
USPC 604/385.25

(57) ABSTRACT

An absorbent sanitary product wearable as a pant-like garment includes a central body including an absorbent portion defining a crotch portion of the product and two side panels extending laterally from the opposite ends the central body to define respective portions of the waist line of the product so that a pair of complementary side panels is provided at each side of the central body. Each side panel has a distal edge connected to a distal edge of the complementary side panel via a refastenable hook-and-loop closure including complementary hook and loop elements. In each pair of complementary side panels one panel carries at its surface facing inwardly of the product either of the hook and loop elements of the hook-and-loop closure. The other panel has its distal edge folded over itself inwardly of the product to form an end loop portion and a marginal rim sandwiched between the two complementary side panels. The marginal rim carries at its surface facing toward the other panel the other of the complementary elements of the hook-and-loop closure.

### CROSS-REFERENCE TO RELATED APPLICATIONS

This application is a continuation of U.S. patent application Ser. No. 12/914,422, filed on Oct. 28, 2010, and published as US 2011/0106042 A1 on May 5, 2011, which claims priority to U.S. Provisional Application No. 61/256,163 filed Oct. 29, 2009, the entire disclosures of which are incorporated herein by reference.

### FIELD OF THE INVENTION

This disclosure relates to absorbent sanitary products.

This disclosure was devised with attention paid to its possible application to absorbent sanitary products that can be worn as pants.

### DESCRIPTION OF THE RELATED ART

For many years, the solution adopted in a practically uniform way by all the producers in the area of absorbent sanitary products has been that of making these products in the form of elements shaped according to a general hourglass conformation, with a central body including an absorbent core designed to collect the body fluids evacuated and two end parts, a front one and a rear or back one. The product is worn by bestowing thereon a general U-shaped conformation and bringing the median stretch of the central body (the so-called “crotch” portion) to extend between the legs of the user.

The end parts extended around. the waist of the user are connected to one another at the homologous side edges, fir example via adhesive tabs, which can normally be repositioned, or similar fastening elements as to be able to re-fasten the product around the body of the user.

These products have traditionally been manufactured and sold in an open condition, i.e., leaving it to the person who applies the product the task of setting it around the body of the user and refastening it according to a general pant-like conformation in the terms referred to previously.

Over the last few years, there has emerged interest in diapers (nappy-pants) of the type commonly referred to as “training pants”. When such a product is taken out of the pack, it has (already) a conformation that substantially resembles that of the pair of pants. It is put on by sliding it over the legs of the user according to criteria basically similar to the ones adopted for putting on pants.

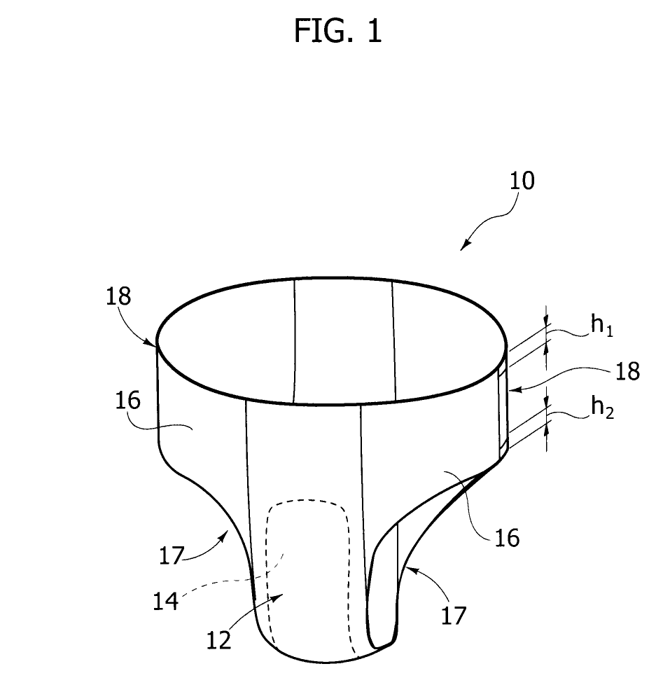

FIG. 1 herein is generally exemplary of the structure of such a “training pant”.

By way of example, in FIG. 1, the reference number 10 indicates as a whole an absorbent sanitary product which can be worn as a pant-like garment, including a central body 12 designed to be applied on the body, of the user by bestowing upon the product a general U shape. In the body 12 there is located an absorbent core 14 which is designed to absorb the body fluids evacuated.

Side panels 16 extend laterally from the central body 12 so as to complete the pant-like configuration of the product 10.

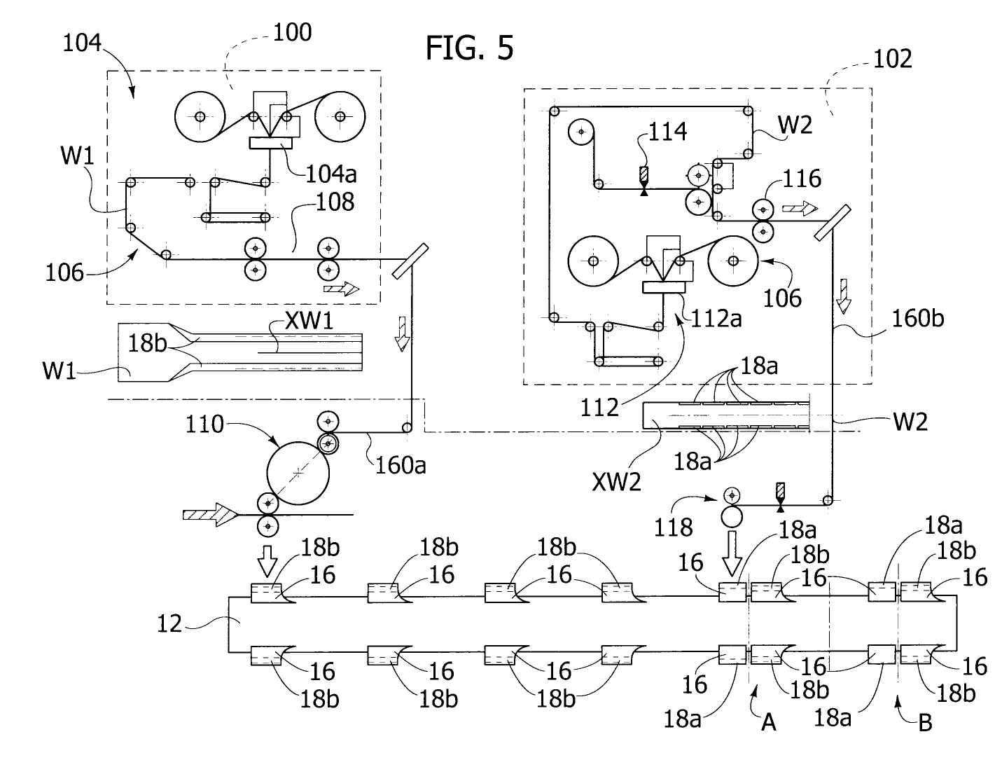

Viewed ideally in an open and distended, condition (i.e. before being closed to a pant-like configuration) the product 10 has, at least approximately, an hourglass conformation as seen at the bottom of FIG. 5 to be described in the following.

The side panels 16 are provided with homologous distal edges 16a designed to be connected (pre-fastened) to one another to form lateral closure regions 18. As better detailed in the following, the pre-fastened closure regions 18 are intended to be re-fastenable, thus permitting the product which is sold in a pre-fastened, closed pant-like condition—to be selectively opened at either side in order to check e.g. whether the product is soiled.

The side panels 16 (which may be elasticated in order to provide a better “fit” of the product around the wearer's body) also include proximal edges 16b that are connected to the central body 12, e.g. by “sandwiching” these proximal edges 16b between the fluid-permeable topsheet 12a and the fluid-impermeable backsheet 12b of the central body 12 and securing it therebetween e.g. via adhesives bonding, ultra sound bonding or the like.

The representation of the product 10 provided in FIG. 1 is of a schematic nature and is intended to highlight the fact that the solution described herein can be applied to a wide variety of possible types of embodiments. Specifically, those who are skilled in the art, while being fully cognizant of e.g. the details related to the choice of the material involved in the production of such a product and the related manufacturing process, will also appreciate that the product 10 may include a wide gamut of additional features including waist-line elastications, elastications of the borders of the openings for the legs of the user (which are designated 17 in FIG. 1), or so-called “cuffs” or elasticated borders arranged along the sides of the absorbent core 14 with the function of providing lateral containment of body fluids. All these details are covered by extensive literature, including patent literature existing on the subject.

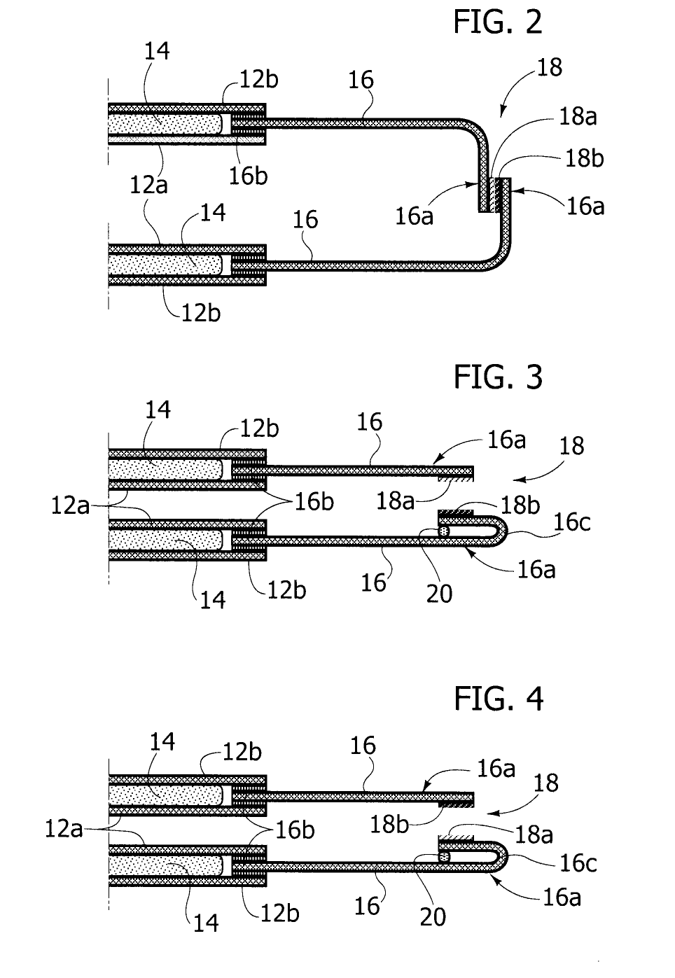

FIG. 2 is schematically representative of a conventional arrangement adopted to provide re-fastenable connection of the distal edges 16a of the side panels 16 in correspondence with the side regions or lines 18.

In the arrangement of FIG. 2 the distal edges 16a of the side panels 16 are connected via a hook-and-loop connection feature (of the type currently referred to as “velcro” closure) including complementary hook-and-loop formations. In certain arrangements, the loop formations designed to operate with the hook formations are simply comprised of the loops formed by the fibres of non-woven layer included in the side panels 16, which may include a multi-layered structure, including e.g. elastic and non-elastic layers intended to be wrinkled due to the elastic action provided by the elastic layers.

Documents such as U.S. Pat. Nos. 6,761,711; 6,849,067; 6,645,190 or 7,534,237 are exemplary of arrangements of the type represented in FIG. 2.

Arrangements as generally represented in FIG. 2 are advantageous in that they facilitate inspection of the product (e.g. to check whether the product is soiled, with possible subsequently refastening), easy removal of the product 10 when soiled, and the possibility of putting on the product without having to remove the shoes of the wearer (typically a baby or a young child), which is particularly appreciated when travelling.

A feature of such arrangements is that (as shown in FIG. 2) the hook portion 18a of the hook-and-loop closure is located at the outer side (so-called “garment side”) of the respective side panel 16, so that the hooks included therein do not run the risk of producing any irritation of the wearer's skin.

Various patent documents such as U.S. Pat. Nos. 7,322,925; 7,335,150; 7,387,148 or EP-A-1 289 465, EP-A-1 289 466, OR EP-A-1 284 700 are exemplary of processes and apparatus which may be applied to manufacturing products are represented in FIG. 2.

### OBJECT AND SUMMARY OF THE INVENTION

Despite the effectiveness of the results obtained, the various arrangements considered in the foregoing have an intrinsic disadvantage in that the associated manufactures processes and apparatus arc inevitably complex, expensive and exposed to criticalities in terms of reliability.

The object of the invention is thus to provide a solution dispensing with these drawbacks while leading to product structure retaining the advantages discussed in the foregoing.

According to the invention, that object is achieved by means of sanitary product having the features set forth in the claims that follow. The invention also relates to corresponding manufacturing process.

The claims are an integral part of the disclosure of the invention as provided here in.

### BRIEF DESCRIPTION OF THE ANNEXED REPRESENTATIONS

The invention will now be described, by way of example only, with reference to the annexed representations, wherein:

FIGS. 1 and 2 have been already described in the foregoing;

FIGS. 3 and 4 are representative of embodiments of a sanitary product;

FIG. 5 is representative of an embodiment of a corresponding manufacturing process; and

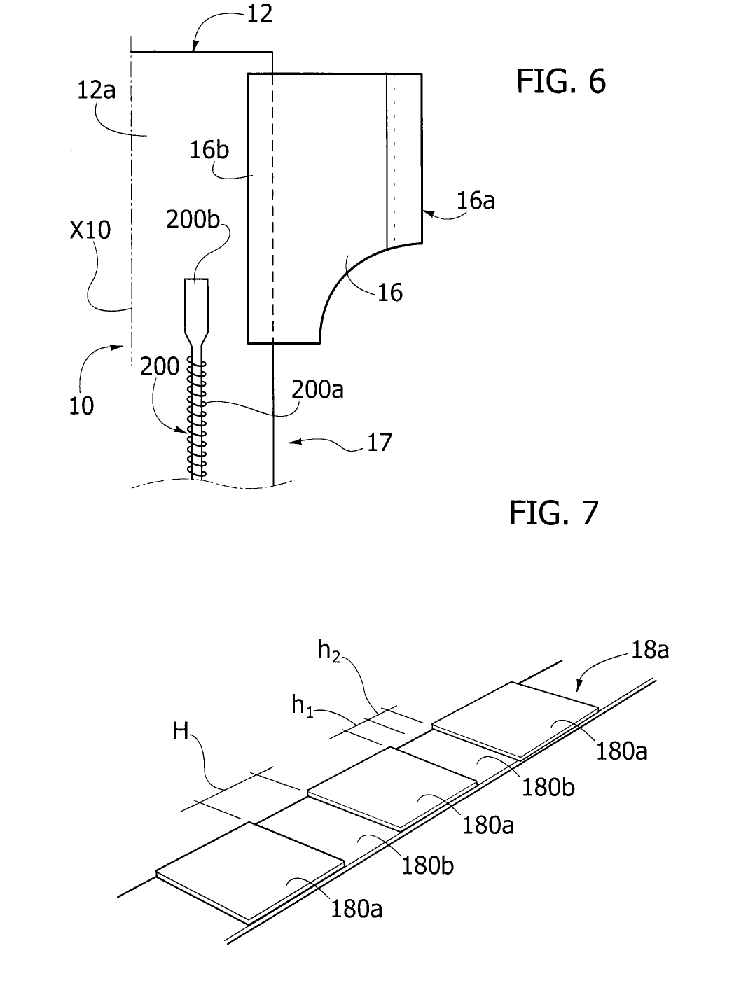

FIGS. 6 and 7 are schematically representative of details of embodiments.

### DETAILED DESCRIPTION OF EMBODIMENTS

In the following description, numerous specific details are given to provide a thorough understanding of embodiments. The embodiments can be practiced without one or more of the specific details, or with other methods, components, materials, etc. In other instances, well-known structures, materials, or operations are not shown or described in detail to avoid obscuring aspects of the embodiments.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The headings provided herein are for convenience only and do not interpret the scope or meaning of the embodiments.

Various embodiments essentially correspond to the general structure of a sanitary product 10 as exemplified in FIG. 1.

Various embodiments disclosed herein may have a number of features in common with the conventional arrangement, e.g. as already described in the foregoing. Again, those who are skilled in the art, while being fully cognizant of e.g. the details related to the choice of the material involved in the production of such a product and the related manufacturing process (unless otherwise described in the following), will also appreciate that the product 10 may include a wide gamut of additional features including waist-line elastications, elastications of the borders of the openings for the legs 17, or so-called “cuffs” or elasticated borders arranged along the sides of the absorbent core 14 with the function of providing lateral containment of body fluids.

Also, in order to avoid rendering this description unduly cumbersome, parts and features which have been already described, or parts or features equivalent to parts and features which have been already described are designated in FIGS. 3 to 7 using the same reference numerals appearing in FIGS. 1 and 2, making it unnecessary to repeat a detailed description thereof.

Various embodiments thus correspond to an absorbent sanitary product 10 wearable as a pant-like garment, including:
a central body 12 including an absorbent portion 14, defining a crotch portion of the product 10, said central body 12 having two opposite ends, and
two side panels 16 extending laterally of said central body 12 at each said opposite ends to define respective portions of the waist line of the product 10, each said side panel (16) having a distal edge 16a connected via a refastenable hook-and-loop closure 18 to a distal edge 16a of a complementary side panel 16 at the opposite end of said central body 12.

FIG. 3 again shows the two “complementary” side panels 16 located at either of the longitudinal sides of the central body 12 having their proximal edges 16b connected to the lateral sides of the central body 12.

As used herein “connected” refers to the joining, bonding, attaching, or the like, of two elements. Iwo elements will be considered to be connected together when they are connected directly to one another or indirectly to one another, such as when each is directly connected to intermediate elements.

In various embodiments, the proximal edges 16b of the side panels 16 are connected to the central body by sandwiching them between the liquid pervious topsheet 12a and the liquid impervious backsheet 12b of the central body 12.

In various embodiments, the side panels 16 may be simply lateral extensions of the topsheet 12a and/or the backsheet 12b of the central body 12.

In various embodiments, at least one side panel 16 in the pair of complementary side panels provided on each side of the central body 12 may be elasticated to provide a better fit of the product around the body of the wearer.

Exemplary arrangements for producing side panels 16 that i.a. are elasticated (either as separate elements connected to the central body 12 or as lateral extensions of the topsheet 12a and/or the backsheet 12b) are disclosed, e.g. in WO 01/91666 A2.

Various embodiments corresponding to FIGS. 3 and 4 provide for connection of the distal edges 16a of the side panels at the region 18 via a refastenable hook-and-loop (i.e. “velcro”) closure, namely a closure including hook formations 18a adapted to engage complementary loop formations 18b.

In various embodiments, the loops of the loop element 18b of the closure may simply be comprised of the loops formed by fibres of an outer surface of the side panels 16. Consequently, while in the figures herein the loop element 18b of the hook-and-loop closure 18 is represented for the sake of clarity and understanding as a distinct feature applied on the side panel 16, those skilled in the art will promptly appreciate that the loop element 18b may be simply provided by surface loops of the side panel 16.

It will be further appreciated that, especially in the views of FIGS. 3 and 4, the relative proportions of parts and elements have been deliberately altered for case of understanding. Also, the complementary side panels 16 illustrated and their distal edges 16a are shown separate (i.e. away) from each other. This is again done for ease of understanding, being otherwise understood that in the product 10 as finally produced, packaged and sold, the complementary side panels 16 abut against each other with their distal edges 16a connected by the hook-and-loop closure 18 to provide the closed condition of the product.

Also, whether the side panel 16 appearing in an upper position in FIGS. 3 and 4 is at the front or back or rear end of the product 10 (“front” and “back” being referred to the condition in which the product 10 is worn by the user) may not be of specific momentum. In various embodiments, such “upper” side panel 16 carrying the hook formations 18a in FIG. 3 and the loop formations 18b in FIG. 4 is designed to be located at the front of the product 10 when worn, while the “lower” side panel, carrying the loop formations 18b in FIG. 3 and the hook formations 18a in FIG. 4 is designed to be located at the back of the product 10 when worn.

By way of contrast with the arrangement of FIG. 2, where the two complementary side panels 16 at each side of the central body 12 have at least approximately the same width or length (i.e. the distance between the distal and proximal edges 16a, 16b), in various embodiments as exemplified in FIGS. 3 and 4, one of the side panels 16 (e.g. the “lower” side panel in the examples of FIGS. 3 and 4) is wider or longer than the complementary side panel (e.g. the “upper” side panel in the examples of FIGS. 3 and 4).

The distal edge 16a of such a longer or wider side panel 16 can thus be folded over itself “inwardly” of the product 10 (i.e. towards the complementary side panel 16) to form an end loop portion 16c. Consequently, the corresponding side panel 16—once folded—will have again approximately the same width or length of the complementary side panel 16 which is not folded. Also, the side edge 16a which is folded over itself will exhibit a terminal rim 16d designed to be sandwiched between the two complementary side panels 16 when the product 10 is closed.

Reference 20 denotes a temporary line of connection (continuous or discontinuous) produced e.g. via a so called “technical” or “green” glue with the purpose of keeping the folded over portion of the distal edge 16d temporarily connected to the inner surface (i.e. the surface intended to face the wearer's body) of the respective side panel 16 in order to avoid that the portion bent may undesirably “flap” with respect to the corresponding side panel 16.

The two side panels 16 will thus be connected at their distal edges 16a via the closures 18 sandwiched therebetween namely via complementary hook-and-loop formations arranged:
at the (inner) surface of the distal edge 16a of the side panel 16 which is not folded over itself (i.e. the side panel 16 shown in the upper position in FIGS. 3 and 4); and
at the surface of the portion of the distal edge 16d which is folded over itself inwardly of the side panel 16 appearing at the lower position in FIGS. 3 and 4.

When the product 10 is worn, the front and rear or back portion of the product (which rest against each other in the product as packaged flat) will be spread apart causing a corresponding spreading apart of the pairs of complementary side panels 16 at each side of the product 10. This will also result in any possible temporary connection as produced by the “technical” or “green” glue 20 being broken, while the complementary elements 18a, 18b will be safely prevented from exerting any negative action on the wearer's skin.

Once the product 10 is worn, the element of the hook-and-loop closure carried by the folded over portion (16e, 16d) will in any case face away from the wearer's body while the element mounted on the inner surface of the other side panel 16 (the one shown in the upper position in both FIGS. 3 and 4), while facing inwardly towards the user body, will in any ease be “masked” and thus shielded with respect to the user's body by both the distal edge 16a of the complementary side panel 16, i.e. the side panel 16 defining the terminal rim 16d, and the other element of the hook-and-loop connection element.

This will apply irrespective of the mutual location of the two elements 18a, 18b, i.e. irrespective of whether:
the hook element 18a is carried by the side panel 16 which is—not—folded, while the loop element 18b carried by the side panel 16 which is folded (as is the case of FIG. 3),
the hook element 18a is carried by the side panel 16 which is folded, while the loop element 18b carried by the side panel 16 which is—not—folded (as is the case of FIG. 4).

Once again, it is recalled that the loop clement 18b will not necessarily be a separate element but may be simply comprised of surface loops of the (e.g. non-woven) structure of the corresponding side panel 16.

FIG. 5 is schematically exemplary of a process and apparatus adapted for producing sanitary products as exemplified in FIG. 3 (and, with minor changes, FIG. 4).

In the representation of FIG. 5, reference number 100 and 102 denote two sources of a two continuous web materials W1 and W2 to be dispensed at 160a and 160b to produce the side panels 16. Irrespective of whether folded or not folded the side panels may be comprised of material produced as disclosed on WO 01/91666 A2, already cited. Various embodiments of the side panels 16 may include the material available under the trade designation of Flex Feel 34795 with the Tredegear company of Richmond, Va. (USA).

As indicated, the exemplary embodiment considered in FIG. 5 refers to the production of sanitary product as exemplified in FIG. 3, namely products where the hook elements 18a are carried by the side panels 16 which are—not—folded while the loop elements 18b are carried by the side panels 16 which are not folded, such loop elements 18b being comprised of surface loops of the structure of the corresponding side panels 16.

Consequently, in the device 100 the material of the side panels 16 whose distal portions 16a carry (i.e. form) the loop elements 18b is first dispensed from a conventional source 104 (e.g. a roll dispenser having associated a splicer 104a to provide continuity of the web W1 dispensed). A folder device 106 cascaded to the source 104 folds over themselves the two longitudinal side edges thereof as schematically shown in FIG. 5 to provide at each side of the web W1 a folded over configuration which produces the folded-over configuration of the side panels 16 appearing at the lower positions in FIGS. 3 and 4.

As shown in FIG. 5, the folder device 106 does in fact forms in the web W1 two complementary portions with mirror-like symmetry with respect to the medina longitudinal axis XW1, each portion designed to give rise to the side panels 16 located at one side (right or left, with reference to the wearer) of the product 10. To that effect the web W1 leaving the folder device 106 is subsequently fed to a cutter unit 108 (of conventional manufacture) which cuts the web W1 in correspondence with its longitudinal median axis XW1 as shown in FIG. 5.

The two mirror-symmetrical sub-webs thus produced are fed at 160a to an applicator unit 110 which performs the following operations:
“segmenting” the two sub-webs to produce therefrom individual side panels 16,
spacing (“repitching”) the side panels 16 thus produced with a pitch corresponding to the length of the individual products 10, and
applying the side panels thus produced and re-pitched to the opposite sides the central bodies 12 of the products 10 which are connected in a continuous chain of adjacent “blanks”.

The overall structure of the device 102 providing the side panels 16 that are not folded is essentially similar in that it again includes a source 112 including a splicer 112a to deliver a continuous web W2. The web W2 is fed to an applicator station 114 where individual “stubs” of hook material 18a are applied (e.g. by glueing, thermal bonding, ultrasound bonding) along the opposite sides of the web W2 as schematically shown in FIG. 5.

The web W2 having the “hook” stubs 18a applied at its sides is fed to a longitudinal cutter device 116 where the web W2 is cut lengthwise along its longitudinal median axis XW2 to produce two mirror-symmetrical sub-webs for feeding at 160b to an applicator station 118, which again performs the operations of:
“segmenting” the two sub-webs to produce therefrom individual side panels 16 carrying the hook elements 18a,
spacing (“repitching”) the side panels 16 thus produced with a pitch corresponding to the length of the individual products 10, and
applying the side panels thus produced and re-pitched to the opposite sides the central bodies 12 of the products 10 which are connected in a continuous chain of adjacent “blanks”.

These blanks completed with the addition of the side panels are subsequently cut as shown at A and B in the lower portion of FIG. 5 to form individual products 10 that are subjected to V- or U-folding at a folding line C longitudinally intermediate A and B.

The V- or U-folding operation performed at C will bring the complementary side panels 16 connected to the opposed ends of each product 10 at each side of the product 10 in a mutually facing position as represented in FIGS. 3 and 4. As a consequence, the distal edges 16a of the two side panels 16 facing each other will abut against each other thus leading to engagement of the two elements 16a, 16b of the hook-and-loop closure 18. The product 10 will thus be closed to the “pre-fastened” condition in which a “training pant” product 10 is packaged and sold.

The basic machinery layout illustrated in FIG. 5 can be easily adapted to produce sanitary products according to FIG. 4, by moving the hook applicator station 114 from the device 102 to the device 100 and arranging it between the folder device 106 and the cutter device 108 so that the hook elements 18a will be applied onto the opposite folded over sides of the web W1.

The system illustrated in FIG. 5 can be assembled with machinery that is conventional in the art.

For instance the sources of the web materials W1 and W2, including the “splicer” units 104a and 112a may be of the type disclosed e.g. U.S. patent publication US2007/0113984.

The folder device 106 may be of any of the types disclosed e.g. in Italian patent IT-B-195 581 or in Italian patent application PE92A000001 or PE2004A000001.

Apparatus adapted to perform the functions of the applicator stations 110 and 118 are disclosed (also in connection with the “repitch” function) in EP-A-1 772 403.

The material comprising the hook formations 18a may be of the type currently available with hook-binder GmbH of Tuttlingen (Germany).

When connection of the elements in question is by adhesive means, adhesives such as PM357 E available with Savarè Specialty Adhesives of Milan (Italy) may be used.

Again, while adhesively connected elements have been considered by way of example, various embodiments may adopt connection via thermal bonding, ultrasound bonding or combinations of the various techniques considered.

Those who are skilled in the art will promptly appreciate that the representation of FIG. 5 is by its nature schematic. Other features of the apparatus and system of FIG. 5 not specifically considered herein are conventional in the art, thus making it unnecessary to provide detailed description herein.

FIGS. 6 and 7 refer to features of various embodiments.

FIG. 6 refers to a sanitary product 10 as exemplified in FIG. 3 shown in an ideal open and extended condition. It will be appreciated that in FIG. 6 only half of the product 10 is shown (i.e. only the half lying at one side of the median longitudinal axis X10 of the product. Also, only one of the end portions (e.g. the back portion) is shown.

FIG. 6 illustrates the possible provision of elasticated bands 200 along of the sides of the central body 12 of the product 10. Each band 200 extends between the complementary side panels 16 located at both ends (front and area or back in the product 10 when worn) with the aim of rendering those portion elastically contractible to provide a closer fit of the product 10 around the legs of the user.

In the embodiment illustrated the elastication band is provided by an elastic material of any known type having a central or intermediate portion 200a which is connected (e.g. by gluing, i.e. by adhesive connection) to the central body 12 of the product 10 in order to provide a desired degree of elastication.

Reference 200b denotes an end portion of the material forming the elastication 200 which extends approximately (lengthwise of the product 10) in correspondence with the side panel 16. Another, symmetrical end portion—not visible in the drawing—is provided at the opposite end of the product 10.

While comprised of a material which is by itself elastic (namely capable of recovering its original size and shape after removal of a force causing a deformation) the end portions 200b are not connected to the central body 12 and thus does not provide any elastication effect proper.

Finally, FIG. 7 illustrates that each stub comprising one hook element 18a as disclosed in the foregoing (and thus the continuous strip of material of which such stubs are produced—see 114 in FIG. 5) may exhibit a discontinuous structure, i.e. be comprised of a plurality of “active” pads 180a (i.e. pads provided with either hook or loop elements of an hook-and-loop closure) separated by flexible intermediate portions 180a that are “passive” (i.e. do not carry either hook or loop elements of an hook-and-loop closure).

FIG. 7 illustrates an exemplary arrangement produce by affixing active hook pads 108a onto a flexible ribbon providing the passive portions 108b therebetween. A similar arrangement may be adopted for the loop formations 18b, by affixing active loop pads 108a onto a flexible ribbon (e.g. a textile ribbon) providing the passive portions 108b therebetween. The active loop portions may also be provide in the form of portions of the flexible ribbon having surface fibres.

The dimensions of the active pads 180a and/or the spacing therebetween, i.e. the dimensions of the passive portions 108b may be either constant or possibly vary depending on specific needs and requirements.

Manufacturing of an element as considered herein may involve simply modifying a current manufacturing process of hook or loop elements of a hook-and-loop closure involving applying/forming hooks or loop formations on a flexible support tape so that hooks (or loops, respectively) are not applied/formed on the “passive” areas 180b, which are thus free of hooks or loops.

Resorting to the arrangement exemplified in FIG. 7 (for either or both of the hook and loop elements) renders the closure formations located at the region 18 more flexible and “soft”, thus increasing the wearer's comfort.

The representation of FIG. 7 shows that the spacing H between pads 108a can be defined as comprised as the sum of two distances h₁, h₂ (namely H=h₁+h₂) where h₁ and h₂ denote the distances (as schematically shown in FIG. 1) separating the ends of each stub 18a from the marginal upper and lower edges of the side panels 16.

This will ensure that once the individual products 10 are separated A and B are shown in FIG. 5 the “active” pads 180a carrying e.g. the hook elements will in any case be located at a distance from the upper and lower edge portion of the side panels, thereby avoiding that the user may be exposed to any “hard” edge.

Of course, without prejudice to the underlining principles of the invention, the details and embodiments may vary, even significantly, with respect to what has been described and illustrated herein merely by way of example, without departing from the scope of the invention as defined by the annexed claim.

1. An absorbent sanitary product wearable as a pant-like garment, including:
a central body including an absorbent portion defining a crotch portion of the product, said central body having two opposite ends, and
two side panels extending laterally from said central body at each said opposite ends to define respective portions of the waist line of the product whereby a pair of complementary side panels is provided at each side of said central body, each side panel of said side panels having a distal edge connected to a distal edge of the complementary side panel at the opposite end of said central body via a refastenable hook-and-loop closure including complementary hook and loop elements:
wherein in each pair of complementary side panels;
a first panel of the panels carries at a surface thereof facing inwardly of the product one of the complementary hook and loop elements of said hook-and-loop closure, said first panel comprising a first distal edge;
a second panel of the panels has a second distal edge thereof folded over itself inwardly of the product to form an end loop portion and a marginal rim, said marginal rim being sandwiched between said first panel and said second panel; and
said marginal rim carries at a surface thereof facing toward said one of the panels the other of the complementary hook and loop elements of said hook-and-loop closure;
wherein a distance from said central body to said first distal edge along said first panel is less than a second distance from said central body to a third distal edge of said end loop- portion along said second panel;
elasticated formations extending at the sides of said central body, each elasticated formation of said elasticated formations located between said two complementary side panels to provide a close fit of the product around the wearer's leg, wherein each said elasticated formation includes:
an intermediate, portion connected to said central body, and
two elasticated end portions extending from said intermediate portion, wherein said end portions extend longitudinally relative to a longitudinal dimension of said central body, said elasticated end portions located on said ends of said central body connected to said two side panels, said elasticated endportions unconnected to said central body, said intermediate portion located on a middle portion of said central body, said middle portion unconnected to said two side panels and located longitudinally between said side panels.

2. The product of claim 1 wherein the hook element of said hook-and-loop closure comprises surface loops of the material comprising the corresponding side panel.

3. The product of claim 1 wherein the second panel carries the loop element of said hook-and-loop closure.

4. The product of claim 1 wherein the second panel carries the hook element of said hook-and-loop closure.

5. The product of claim 1 wherein said marginal rim sandwiched between the two complementary side panels is temporarily connected to the corresponding side panel, the temporary connection releasable when the product is opened up to be worn.

6. The product of claim 1 wherein at least one side panel in said pair of complementary side panels is elasticated.

7. The product of claim 2 wherein folded-over side panel carries the loop element of said hook-and-loop closure.

8. The product of claim 2 wherein folded-over side panel carries the hook element of said hook-and-loop closure.

9. The product of claim 2 wherein said marginal trim sandwiched between the two complementary side panels is temporarily connected to the corresponding side panel, the temporary connection releasable when the product is opened up to be worn.

10. The product of claim 3 wherein said marginal rim sandwiched between the two complementary side panels is temporarily connected to the corresponding side panel, the temporary connection releasable when the product is opened up to be worn.

11. The product of claim 4 wherein said marginal rim sandwiched between the two complementary side panels temporarily connected to the corresponding side panel, the temporary connection releasable when the product is opened up to be worn.

12. The product of claim 2 including elasticated formations extending at the sides of said central body. each elasticated formation extending between two said complementary side panels to provide a close fit of the product around the wearer's leg, wherein each said elasticated formation includes:
an intermediate portion connected to said central body, and
two end portions extending away from said intermediate portion, wherein said end portions extend in correspondence with said side panels, said end portions being exempt from connection with said central body.

13. The product of claim 3 including elasticated formations extending at the sides of said central body, each elasticated formation extending between two said complementary side panels to provide a close fit of the product around the wearer's leg, wherein each said elasticated formation includes:
an intermediate portion connected to said central body, and
two end portions extending away from said intermediate portion, wherein said end portions extend in correspondence with said side panels, said end portions being exempt from connection with said central body.

14. The product of claim 4 including elasticated formations extending at the sides of said central body, each elasticated formation extending between two said complementary side panels to provide a close fit of the product around the wearer's leg, wherein each said elasticated formation includes:
an intermediate portion connected to said central body, and
two end portions extending away from said intermediate portion, wherein said end portions extend in correspondence with said side panels, said end portions being exempt from connection with said central body.

15. The product of claim 5 including elasticated formations extending at the sides of said central body, each elasticated formation extending between two said complementary side panels to provide a close fit of the product around the wearer's leg, wherein each said elasticated formation includes:
an intermediate portion connected to said central body, and
two end portions extending away from said intermediate portion, wherein said end portions extend in correspondence with said side panels, said end portions being exempt from connection with said central body.

16. An absorbent sanitary product wearable as a pant-like garment, including:
a central body including an absorbent portion defining a crotch portion of the product, said central body having two opposite ends, and
two side panels extending laterally from said central body at each said opposite ends to define respective portions of the waist line of the product whereby a pair of complementary side panels is provided at each side of said central body, each side panel of said side panels having a distal edge connected to a distal edge of the complementary side panel at the opposite end of said central body via a refastenable hook-and-loop closure including complementary hook and loop elements;
wherein in each pair of complementary side panels;
a first panel of the panels carries at a surface thereof facing inwardly of the product one of the complementary hook and loop elements of said hook-and-loop closure, said first panel comprising a first distal edge;
a second panel of the panels has a second distal edge thereof folded over itself inwardly of the product to form an end loop portion and a marginal rim, said marginal rim being sandwiched between said first panel and said second panel; and
said marginal rim carries at a surface thereof facing toward said one of the panels the other of the complementary hook and loop elements of said hook-and-loop closure; and
wherein a third distal edge of said end loop portion extends further away from said central body than said first distal edge when said second distal edge is folded over and said one of the hook and loop elements is connected to said other of the hook and loop elements.

17. The product of claim 1 wherein said first panel comprises a front panel and said second panel comprises a rear panel, said front panel and said rear panel being relative to a front and a rear of a user.
